SEQUENCE LISTING (1 sequence)

SEQ ID NO: 1 - DNA, 10 bp, human
ggggttgggg

(19) United States
(12) Patent Application Publication
(10) Pub. No.: US 2004/0110163 A1
(43) Pub. Date: Jun. 10, 2004

(54) Organic nanoeletric conductors

(76) Inventors: Alexander Kotlyar, Zoran (IL); Danny Forath, Jerusalem (IL); Miron Hazani, Nir-Israel (IL)

Correspondence Address: Eitan Pearl Latzer & Cohen Zedek, Suite 1001, 10 Rockefeller Plaza, New York, NY, 10020, US

(21) Appl. No.: 10/362,443

(22) PCT Filed: Sep. 6, 2001
(86) PCT No.: PCT/IL01/00845

(51) Int. Cl. H01L 21/00; C12Q 1/68

(52) U.S. Cl. 435/6; 438/1

(57) ABSTRACT

The present invention relates to nanoelectric conductors and, more specifically, to conductive organic molecules capable of electron transport for use in biosensors and other types of electronics, including semi-conductors, transistors and switches.

### FIELD OF THE INVENTION

The present invention relates to molecular nanoelectric conductors and, more specifically, to conductive organic molecules capable of electron transport for use in biosensors and other types of electronics, including semi-conductors, transistors and switches.

### BACKGROUND OF THE INVENTION

Current interest in the field of molecular scale electronics prompted the search for organic molecules capable of efficient electron transport to be used as nanoelectric conductors.

Long range electron transfer reactions involve the quantum mechanical tunneling of electrons from a donor to an accepter group separated by relatively large (more than 20) molecular distances. The semi classical Marcus theory for non-adiabatic processes is commonly used for interpreting the measured long-range electron transfer rates. (Marcus, R. A. (1965) J. Chem. Phys. 43, 679-701 and Marcus, R. A. and Sutin, N. (1985) Biochem. Biophys. Acta 811, 265-322). According to Marcus theory, the rate of a non-adiabatic outer-shell electron transfer between adjacent reactants is given by the following equation:

kₑₗ=10¹³ exp(−β(d−3))exp(−λ+ΔG^{o})²/4λRT)

where d is the distance between the redox active centers, β is a scalar that correlates the rate with separation distance (d), ΔG^{o} is the standard free energy of the reaction and λ is the reorganization energy. The first exponent of equation 1 expresses the effect of the distance (d) factorized by β on the reaction's rate. A major focus of current research in biological electron transfer is directed towards understanding how distance and structure of the intervening medium between redox active centers influence the electron transfer rates.

Some researchers have suggested that DNA may support efficient long-range electron transfer. According to this view a double helical DNA molecule can be treated as a π stacked conductivity system which allows electrons to move effortlessly as a current through an electrical wire.

There is currently a heated debate whether DNA can mediate long range electron transfer. On the one hand, scientists at the California Institute of Technology, headed by Professor Barton, have presented data supporting the unique conductive properties of DNA. (Murphy, C. J., Arkin, M. R., Jenkins, Y., Ghatlia, N. D., Bossmann, S. H., Turro, N. J., and Barton J. K. (1993) Science 262, 1025-1028) On the other hand, quantum mechanical calculations by Beratan, state that the electronic coupling in a double helical DNA molecule drops rapidly with distance, resulting in its poor conductivity. (Beratan, D. N. (1996) J. Phys. Chem. 100, 17678-17682) At present there is no satisfactory evidence for efficient conductive properties of single or double helical DNA.

Recently a high resolution crystal structure of a guanine tetraplex adopted by d(TGGGGT) in the presence of sodium ions was obtained (Laughlan, G., Murchie, A. I. H., Norman, D. G., Moore, H. I., Moody P. C., Lilley, D. M., and Luisi, B. (1994) Science 265, 520-523) The general topology is of a right handed helix composed of 4 equivalent strands with a helical repeat of 10.4 terads per turn. The strands associate by formation of hydrogen bonded guanine tetrads and are stabilized by coordination of the sodium ions with the O-6 carbonyl oxygens. Two modes of coordination are available, tetra coordination in the center of the planar array of the guanine tetrads and octa coordination on the helical axis but midway between the tetrads.

Naturally occurring quartet structures are usually stabilized by physiological abundant ions such as potassium and sodium yet a variety of mono and divalent ions can stabilize these structures. There is a correlation between the ionic radius of the coordinated ions and their stabilizing effect such as K+, NH4⁺ (1.33A)>Ca+² (0.99 A)>Na⁺(0.97 A)>Li⁺(0.68 A)>Mg⁺²(0.66 A). (Hardin, C. C., Henderson, E., Watson, T., and Prosser, J. K. (1991) Biochemistry. 30, 4460-4472)

Recently, electron transfer rates constants from guanine multiplets were measured by cyclic voltametry of ruthenium bipyridine complexes. This technique is based on the oxidation of guanine nucleotides located at various distances from an oxidizing moiety. These results have shown that guanines in G quartets are only slightly more active in electron transfer than guanine nucleotides in regular B DNA. (Sistare, M. F., Codden, S. J., Heimlich, G., and Thorp, H. (2000) J. Am. Chem. Soc., 122, 4742-4749) It thus seems that double helical DNA as well as sodium and potassium stabilized G quartet structures cannot support long range electron transfer.

Szalai et al recently disclosed an analysis of the electron transfer properties of tetrads, concluding that adjacent guanines in such tetrads are not hole traps in guanine quartets (Szali, V. A. and Thorp. H. H. (2000) J. Am. Chem. Soc 122, 4524-4525). The paper concluded that guanines in guanine quartets are slightly more accessible to a Ru(III) oxidant than in B-form duplexes, but that an increase in reactivity due to stacking of adjacent guanines does not occur in the guanine quartet. This demonstrates the present direction of research to the conductivity of the guanine quartets per se, which is unrelated to the conductivity of the coordinated ions trapped therein.

### SUMMARY OF THE INVENTION

The present invention provides a polymeric conductive wire comprising nucleic acids forming a central canal composed of linearly arranged metal ions within the canal and an electron-rich external surface. The present invention provides a molecular conductive wire comprising nucleic acids forming a central canal composed of linearly arranged metal ions within the canal and an electron-rich external surface. In another embodiment, the wire includes a non-conductive outer organic portion and an internal core of redox active ions.

The present invention relates to the novel finding that redox active ions disposed in the internal cores of guanine tetraplexes and coordinating such tetraplexes are capable of electron transfer and thereby capable of conductivity as organic wires. Such organic wires can be used in semi-conductors, transistors, and switches. In accordance with the present invention, there is provided a molecular conductive organic wire. More specifically, the wire includes a non-conductive outer organic portion and an internal core of redox active ions.

The present invention further provides a method of producing a molecular conductive wire by simultaneously forming nucleic acids tetraplexes hydrogen bonded together and stabilizing the wire through coordinated redox active ions disposed in a core of at least some of the tetraplexes. In one embodiment, the nucleic acids are guanine.

The present invention also provides a switch for controlling the flow of electrons through the conductive organic wire. The present invention also provides a transistor comprising the molecular conductive wire.

The present invention additionally provides a micro-array and for the fabrication thereof, having a support and series of conductive organic wires operatively connected to each other.

### DETAILED DESCRIPTION OF THE DRAWINGS

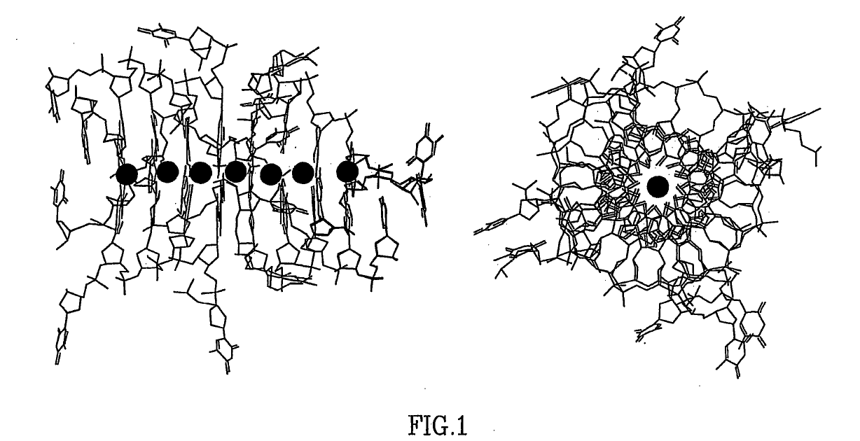

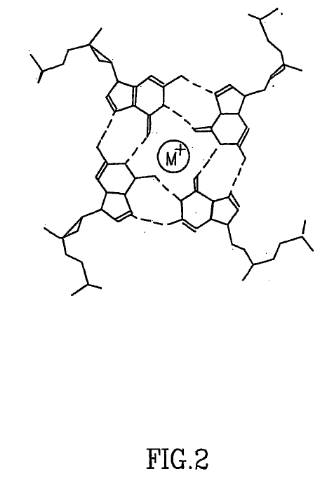

FIG. 1. The crystal structure of a guise tetraplex.

FIG. 2. Schematic presentation of chemical structure of G-quartet M+-alkali metal cation.

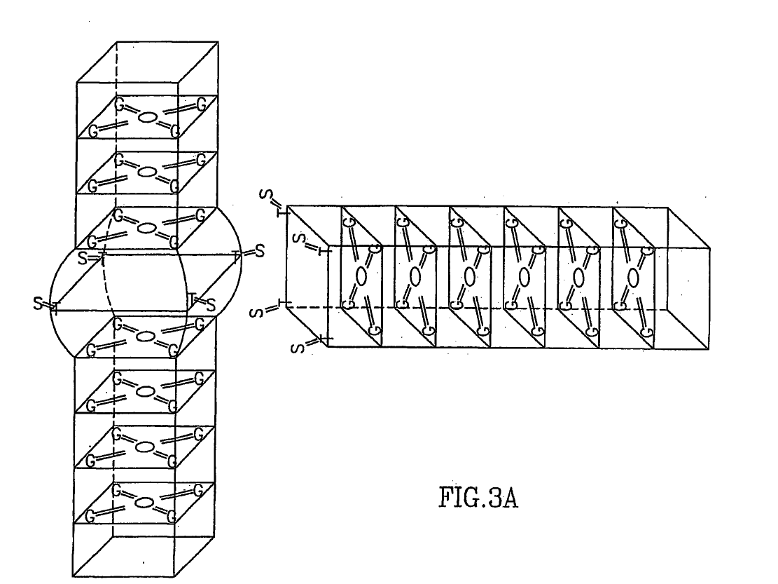

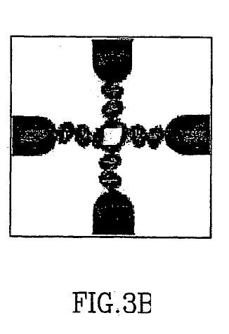

FIG. 3. A. Schematic presentation of chemical structure of the transistor comprising G-quartet metal cation B. Schematic of a simple DNA network connected to a set of 4 nanofabricated electrodes.

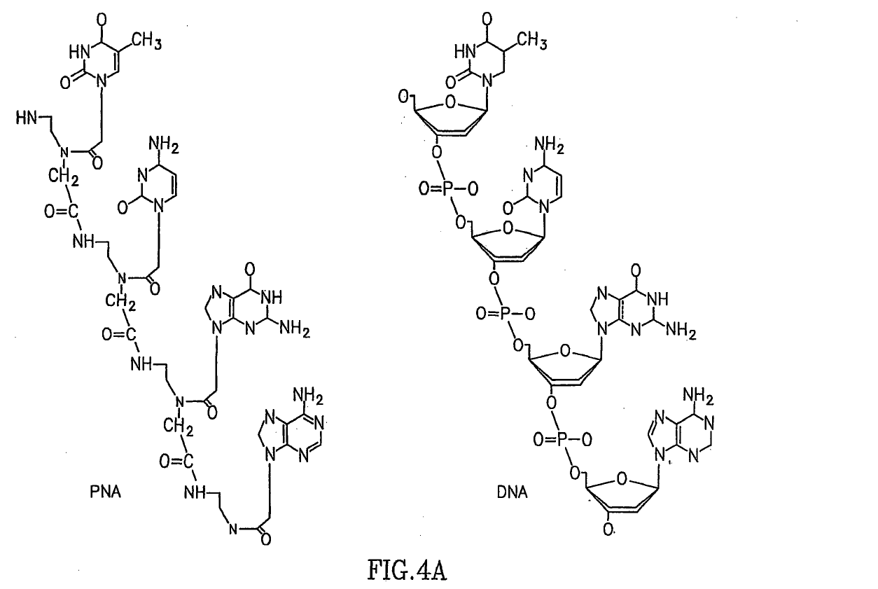

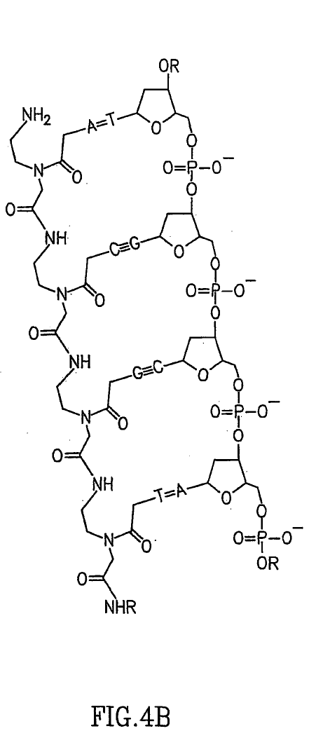

FIG. 4. A. Chemical structures of DNA and PNA. The deoxyribose phosphodiester backbone in DNA has been changed to N-(2-aminoethyl)glycine in PNA. B. Specific duplex structure formed between complementary PNA and DNA strands.

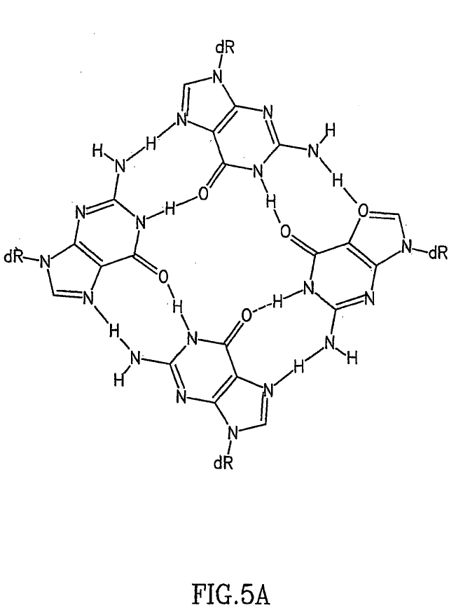

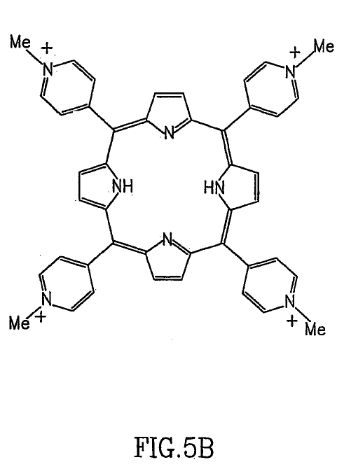

FIG. 5. A. Structures for individual guanine base G-tetrad, showing H-bonded planar arrangement, and B. H2TMyP cationic porphyrin.

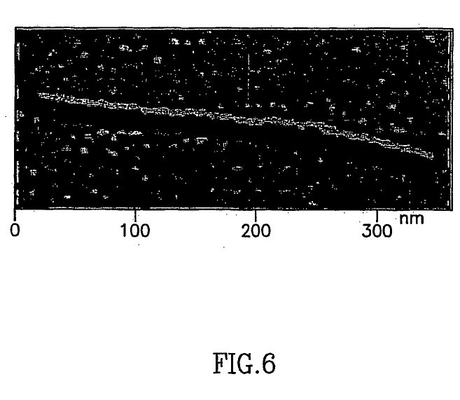

FIG. 6. Presents an STM picture of a G4-DNA wire made of 30G-oligonucleotide.

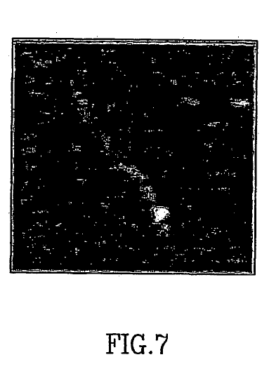

FIG. 7. An AFM image of a 400 nm long single G4-DNA wire.

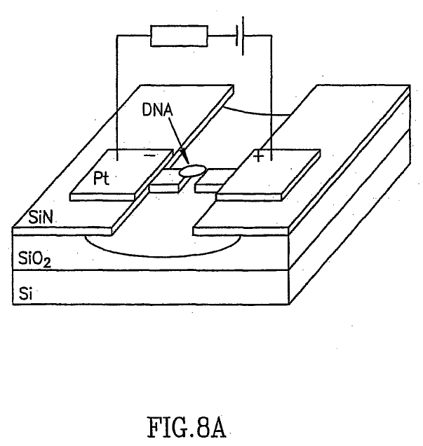

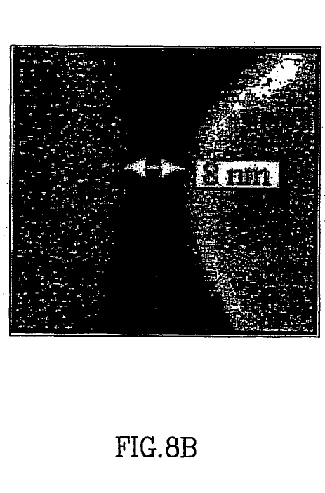

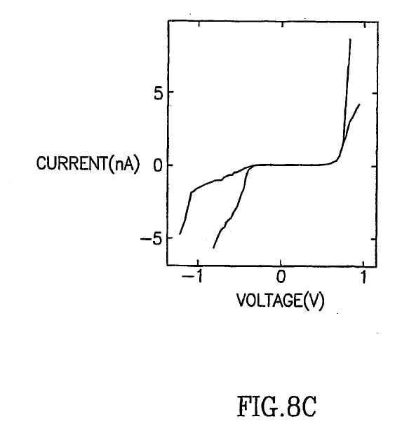

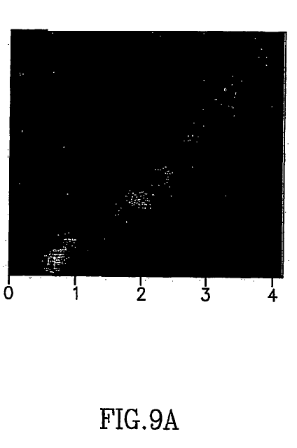

FIG. 8. A. Schematic of a nanofabricated device, B. a scanning electron micrograph of the electrodes; and C. I-V curves measured through DNA.

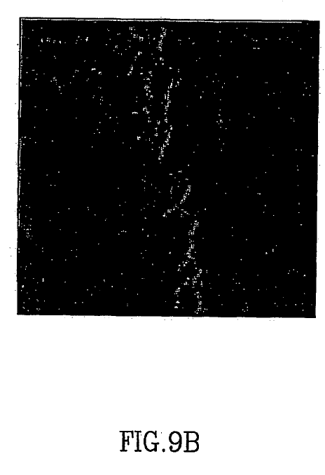

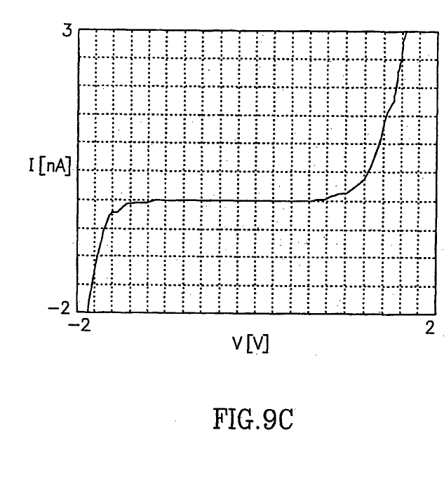

FIG. 9. A STM image of a single DNA molecules; B. a supercoiled DNA; and C. current-voltage characteristic measured on a DNA molecule.

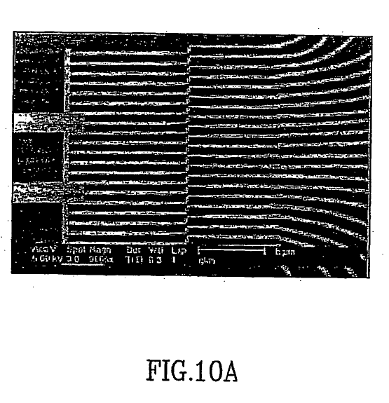

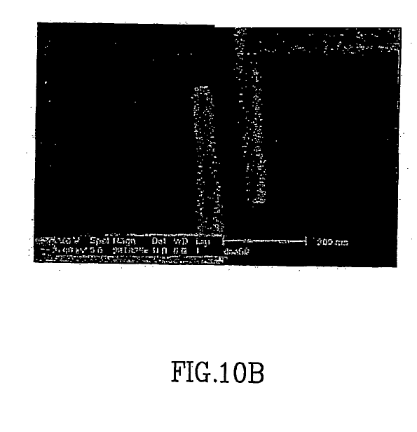

FIG. 10. A. Scanning probe microscopy image of an example of a planar arrangement of a set of metal electrodes on an insulating substrate; and B. one pair of the electrodes.

### DETAILED DESCRIPTION OF THE INVENTION

The present invention provides a polymeric conductive wire comprising nucleic acids forming a central canal composed of linearly arranged metal ions within the canal and an electron-rich external surface. The present invention provides a molecular conductive wire comprising nucleic acids forming a central canal composed of linearly arranged metal ions and an electron-rich external surface cent. The present invention provides a conductive organic wire. In another embodiment, the wire includes a non-conductive outer organic portion and an internal core of redox active ions.

The present invention relates to the novel finding that redox active ions disposed in the internal cores of guanine tetraplexes and coordinating such tetraplexes are capable of electron transfer and thereby capable of conductivity as organic wires. Such organic wires can be used in semi-conductors, transistors, and switches. In accordance with the present invention, there is provided a molecular conductive organic wire. More specifically, the wire includes a non-conductive outer organic portion and an internal core of redox active ions.

In one embodiment, the organic wire is a molecule comprising polynucleotides based nanoelectronic conductive wires. In one embodiment, the backbone of the molecular wire is comprised of nucleotides, proteins, or lipids or a combination thereof. In one embodiment, the backbone of the molecular wire comprises a DNA, RNA, M-DNA, or PNA. In another embodiment the organic wire is a DNA-based nanoelectronic conductive wires. In another embodiment the organic wire is a DNA-mimetic based nanoelectronic conductive wires. In another embodiment the organic wire is a RNA-based nanoelectronic conductive wires. In one embodiment the DNA is a G4-polynucleotide, which is a four-stranded stable structure that contains a relatively planar arrangement of four guanine (G) nucleotides. In one embodiment the DNA is a G4-DNA. In one embodiment the DNA is a G4-RNA.

In another embodiment the DNA is a G4-DNA and C4-DNA combination which is a four-stranded stable structure that contains a planar arrangement of four cytosine (C) nucleotides. In another embodiment the DNA is a C4-DNA, which is a four-stranded stable structure that contains a planar arrangement of four cytosine (C) nucleotides.

These organic wires are based on complexes of G4-DNA tetramolecular parallel quadruplex and also G4-DNA with metal-ions in the central space of each G4 plane. In one embodiment, the electronic conductivity of G-quartet based wires is altered and controlled by changing the nature of metal ions in the wire and the average distance between the neighboring redox-active metal ions. In one embodiment the major G quartet structures is a G4-DNA: tetramolecular parallel quadruplex. In another embodiment, the G′4-DNA: unimolecular anti-parallel quadruplex. In another embodiment the G′2-DNA: biomolecular anti-parallel quandruplex.

As defined herein a G4-DNA is a DNA and RNA containing consecutive G bases can form extensive four-stranded conformations based on the hydrogen-bonded guanine tetrad, or G-quartet, as shown by X-ray crystallography. FIG. 1 below depicts top and side views of the crystal structure of a G4-DNA in the presence of sodium ions (black circles enlarged to simulate the ionic radius). The ions are arranged in a linear manner separated by an average distance of from 3.0 to 4.0 A. In another embodiment the ions are arranged in a linear manner separated by an average distance from 3.2 to 3.8 A. In another embodiment the ions are arranged in a linear manner separated by an average distance from 3.4 to 3.6 A. In another embodiment the ions are arranged in a linear manner separated by an average distance of 3.6 A In another embodiment the distance between the neighboring Me-ions is from 3.2-3.6. In another embodiment the distance between the neighboring Me-ions is 3.4.

In one embodiment, as shown in FIG. 1, structurally a G-quartet comprises four G's in a square plane array as shown in FIG. 2 below. Each G is both the donor and acceptor of the two hydrogen bonds. Electronegative carbonyl oxygen atoms line the center of the G-ring where they interact with suitable size cations.

In one embodiment, the poly-G is a On oligonucleotide wherein n=3 or more, in the presence of monovalent and/or divalent ions results in a formation of large superstructures. In one embodiment, the poly-G is a Gn oligonucleotide wherein n=5 or more, in the presence of monovalent and/or divalent ions results in a formation of large superstructures. In one embodiment, the poly-G is a Gn oligonucleotide wherein n=6 or more, in the presence of monovalent and/or divalent ions results in a formation of large superstructures. In one embodiment, the poly-G is a Gn oligonucleotide wherein n=7 or more, in the presence of monovalent and/or divalent ions results in a formation of large superstructures. In one embodiment, the poly-G is a Gn oligonucleotide wherein n=10 or more, in the presence of monovalent and/or divalent ions results in a formation of large superstructures. In one embodiment, the poly-G is a Gn oligonucleotide wherein n=30 or more, in the presence of monovalent and/or divalent ions results in a formation of large superstructures. Incubation of the oligonucleotides at high concentrations (>2 mM) in the presence of monovalent cations yielded a mixture of G4-wires, with different lengths. Application of gel exclusion chromatography enabled to separate the mixture into fractions, having different molecular sizes, up to 0.5 micron.

In one embodiment, the wire comprises different lengths of poly-G DNA as well as poly-G that include tymidine (T) nucleotides in specific locations are made. For example, in one embodiment, the poly-G is a GGGGTTGGGG oligonucleotide in the presence of monovalent and/or divalent ions results in a formation of large superstructures. In one embodiment, the poly-G is a GnTn oligonucleotide wherein Gn=3 or more and T=2 or more, in the presence of monovalent and/or divalent ions results in a formation of large superstructures. In one embodiment, the poly-G is a Gn oligonucleotide wherein n=5 or more and T=2 or more, in the presence of monovalent and/or divalent ions results in a formation of large superstructures. In one embodiment, the poly-G is a Gn oligonucleotide wherein n=6 or more and T=2 or more, in the presence of monovalent and divalent ions results in a formation of large superstructures. In one embodiment, the poly-G is a Gn oligonucleotide wherein n=7 or more and T=2 or more, in the presence of monovalent and/or divalent ions results in a formation of large superstructures. In one embodiment, the poly-G is a Gn oligonucleotide wherein n=10 or more and T=2 or more, in the presence of monovalent and/or divalent ions results in a formation of large superstructures. In one embodiment, the poly-G is a Gn oligonucleotide wherein n=30 or more and T=2 or more, in the presence of monovalent and/or divalent ions results in a formation of large superstructures. In one embodiment, the poly-G is a GGGGTTGGGG oligonucleotide in the presence of monovalent and/or divalent ions results in a formation of large superstructures

In one embodiment, the molecule comprises a Peptide Nucleic Acid (PNA) (see FIG. 4) which is a DNA mimic based on an entirely artificial, acyclic and achiral neutral amide backbone. In this nucleotide analogue the sugar backbone has been replaced by N-(2-aminoethyl)-glycine units but the bases nevertheless form a specific duplex structure with complementary DNA sequences.

The PNA-DNA duplexes that were analysed in solution exhibited a very high thermal stability due to the missing interstrand repulsion between the DNA phosphate groups and the uncharged peptide backbone of the PNA molecules. The Tm of PNA/DNA duplexes is reported to increase at physiological conditions by ˜1.5° C. per base pair compared to that of the equivalent DNA-DNA hybrid. The ability to form a stable PNA-PNA duplexes (their stability is even higher that that of PNA-DNA) permits the production of a stable 4G-PNA structures and complexes of 4G-PNA with various cations. The advantage of using PNA compared to DNA is higher stability of the PNA-based structures and the absence of electrostatic binding of cations to the negatively charged phosphate groups of DNA. The PNA-DNA and PNA-PNA duplexes characterized by very high stability due to the mussing interstrand repulsion between the DNA phosphate groups and the uncharged peptide backbone of the PNA molecules. High stability of the duplexes is an advantage, which permits production of a stable M-configuration of the polymers with various cations.

Further, in another embodiment, the DNA, and a metal free tetracationic porphyrin (H₂TMPyP) and its Zn-derivative (Zn-TMPyP) which molecular dimensions resemble those of a G-tetrad are capable of interactive stacking with the 4G-DNA (see FIG. 5). The mechanism of the porphyrins binding includes intercalation of the ligand between each pair of successive G-tetrad planes in 4G-DNA The planar H₂TMPyP ligand molecule can be intercalated within 4G-DNA and stabilize the complex through favorable π-stacked interactions between aromatic residues, as for “classical” duplex intercalation. The π-stacking between the G-tetrads and the porhyrin within the 4G-DNA-porphyrin complex will induce high conductivity of the structure. Thus by changing the ratio between the 4G and the porphyrin it is possible to alter the conductivity of the polymer. A metal-free and Zn-porphyrins are photoactive and characterized by long lasting excited triplet state. The triplet state characterized by low redox potential and is capable of abstracting an electron at electrical potentials much lower than the ground state. This property of the porphyrins results in the molecules having photo-initiators properties for electrical reactions (electro-optical switches). When complexed with 4G DNA the intercalated porphyrin molecules will carry the current through the polymer at relatively low applied electrical potentials only in the presence of light.

In another embodiment, the DNA backbone is a B-DNA. In another embodiment, the DNA backbone is in an M-DNA configuration. M-DNA is a B-DNA that was modified to contain metal ions like zinc in its core along the hydrogen bonds. At high pH and high metal ion concentration the imino protons of the G and T bases are replaced by metal ions. In another embodiment, the PNA backbone is in M-configuration with various cations.

In another embodiment, the organic portion includes aligned plates, preferably of guanine tetraplexes each having a central space defining an internal core. At least some of the internal cores chelate the redox active ions. The four electronegative carbonyl oxygen atoms compose the hydrophilic central core of the G-ring (in one embodiment with a range of 4-0-5.0 A; in another embodiment with a range of 42-4.8 A; in another embodiment with a range of 4-5-4.7 A; in another embodiment with a diameter of 4.7 A) where they interact with suitable size cation-ion, one per each tetrad. The dipole-charge interactions stabilize complex between the carbonyl oxygens and the metal ion.

As provided herein, conducting organic wires were made on the basis of G4-DNA complexe with redox active metal-ions. The organic wire include the external surface of the molecule and a central canal, composed of linearly arranged metal ions. In one embodiment, the meta ions are selected from a group including, but not limited, to silver, copper, zinc, iron europium, chromium, mercury and ruthenium. The wire may comprise one or more metal ion which are the same or different. The ability to incorporate metal ions with differen contributions to the conduction properties into a single molecular wire permit the wire composed of segments with high and low resistance regions along the conductor. This allow for the manipulation of the conductivity of the wire in order to form nanoelectronic devices.

In one embodiment, the average distance between conducting metal ions along the molecule is controlled by the addition of one or more redox “silent” metals (sodium, potassium) into the central core of the G-quartet together with the one or more redox active ones.

In one embodiment, increasing the distance between conducting metal ions results in lower electronic conduction of the G4-DNA. In another embodiment, the combination of high and low resistance segments along the molecule enables manipulation of the conductivity of the wire and create nanodevices from these molecular wires. For example, the nanodevices can be composed of special segments that form quantum dots, e.g. segments with high metal ion concentration, flanked by tuneling barriers, or designed using the sequence dependence measurements. Furthermore a number of G4- or M-DNA molecules will act as transistors and. For example, if the G4-DNA molecule conducts current from a source to a drain then another G4-DNA molecule can be connected to a third terminal in a T-shape and gate the conduction.

The redox active ions are selected from the group including, but not limited to, silver, copper, iron, europium, chromium, mercury, and ruthenium. It should be noted that this list is not exhaustive, but rather exemplary. The wire may comprise of one or more redox active ions which may be the same or different.

The molecular wire can include one or more spacing tetraplexes, such as thymine tetraplexes, for increasing resistance to electron flow through the wire. The spacing tetraplexes do not coordinate ions thereby spacing apart otherwise conductive redox active ions. Alternatively, non-conductive or inert metals can be disposed in at least some of the guanine tetraplexes effectively spacing conductive redox active ions. This can be accomplished by various means known in the art, such as by combinatorial chemistry. For example, in spontaneously forming the wires of guanine tetraplexes, a mixture of ions can be used pursuant to combinatorial chemistry methods well known in the art. In this manner, a known proportion of conductive and non-conductive ions can be incorporated into tetraplexes providing desired resistance, depending upon the ratio of conductive to non-conductive ions.

The molecular wires of the present invention can include one or more operative interconnections to other conductive molecular wires. Such interconnections can include covalent linking molecules for covalently connecting an end plate of a guanine tetraplex to a tetraplex of a second conductive organic wire. For example, linking can be provided through oxidized thiol groups. Alternatively, amino modified nucleotides can be attached to the 5′ phosphates using standard phosphoramidate chemistry. Alternatively, DNA can be reacted with carbonyldiimidazole and a diamine to yield a phosphoramidate that has a free primary amine. This amine can then be reacted with nucleotides modified with amino reactive groups.

Switches, organic or inorganic, can be incorporated into the wire for controlling electron flow between redox active ions by either allowing or absolutely stopping the flow of electrons. For example, such switching mechanisms can include molecules capable of reversibly intercalating between adjacent plates of guanine tetraplexes, as well known in nucleotide art. Thus, the electron flow can be controlled by light irridation, heat exposure, changes in the ionic environment, or by any environmental change that can alter the redox state of an ion. Thus, any method of changing the redox potential of an ion coordinated in a guanine tetrad complex is sufficient to achieve the desired results. For example, zinc or ruthenium can be coordinated into She tetraplexes, these ions being capable of changing redox potential upon irridation. Exposure to light will change the redox potential of the ions and the conductivity of the wire. Thus, light can be used to turn on and off current and provide a switch mechanism. Likewise, heat or other ambient conditions can alter current flow, thereby providing a switch mechanism.

As previously stated, a number of ions other than sodium and potassium are capable of stabilizing quartet structures. It is highly reasonable that redox active ions having ionic radii similar to potassium such as silver, gold and rubidium can coordinate to the O-6 carbonyl oxygens and thus stabilize the quartet structures. In this case a linear arrangement of redox active ions in very close proximity is formed. The almost Van der Waals contact between the ions ensures strong electronic coupling and the reorganization energy for self-exchange is minimal. Also the crystal field imposed by the coordination sphere will lift the degeneracy of the ion energy levels and create a conduction band. A linear arrangement of closely separated redox active ions leads to the formation of an effective molecular wire by means of electron transfer or transport relating to super exchange or conduction band respectively.

The conductive properties of the suggested bio-electrical molecular “nanowire” depend strongly on the nature of the metal in the complex and on the average distance between the metal ions in the tetra-G structure. The distance between the metal ions can be varied by either changing the nucleotide to ion ratio or by inserting non guanine nucleotides in desired locations along the tetra G complex. Isolated tetrads of nucleotides other than guanine creates local deformations in the quadruplexes that cannot support proper ion coordination thus creating local gaps along the “nanowire”. These gaps are in effect regions with lower electronic conduction. The conductive parameters of the “wire” can be flirter manipulated by locally changing the metal composition. The ability to incorporate a number of metal ions, having different electron conducting properties into a single DNA “wire” enables construction of DNA-metal complexes composed of high and low resistance regions along the conductor. This allows for manipulation of the micro-conductivity of the “nanowire.”

The electrical properties of G4 and M-DNA molecules using SPM and electrical transport are measurement through single molecules attached to nanofabricated electrodes. The current-voltage characteristics are determined in the same fashion as for canonical double stranded DNA molecules and between planar electrodes that are fabricated on an insulating surface (SiO₂ or SiN₄). These measurements are performed at temperatures ranging from room temperature down to cryogenic temperatures and at various environmental conditions (air, solvent and vacuum).

This invention provides for sophisticated molecular electronic heterostructures comprising special segments that form quantum dots, e.g. segments wit high metal ion concentration, flanked by tunneling barriers, or designed using the sequence dependence measurements. Furthermore, this invention provides for a number of G4- or M-DNA molecules which act as transistors (see FIG. 3A) and networks (see FIG. 3B). For example, in one embodiment, the G4-DNA molecule can be connected to a third terminal in a T-shape and gate the conduction.

This invention provides a DNA-based wire-network in contact with or to a frame of nanofabricated planar and freestanding multiple electrodes by electrostatic trapping or self-assembly in preferred arrangements. In one embodiment, the heterostructures comprising a cross-shaped DNA molecule are attached to or in contact with a frame of multiple nanofabricated electrodes using the double-strand recognition property of the DNA.

The above DNA structures and networks are manufactured as previously described (Seeman, N. C. and Kallenbach, N. R. (1994) Annu. Rev. Biophys. Biomol. Struct. 23, 53; Eichman B. F., Vargason, G. M, Mooers B. H. M. and Ho P. S. (2000) PNAS 97, 3971. The accuracy of the assembly are controlled using polyacrylamide gel electrophoresis. Samples are run on the acrylamide gel against different combinations of the four different oligonucleotides used to generate the specific structure. The simplest forms of such networks are flexible three arms junctions, the stiffer four arms structures and H-like configurations. Cross-like structures are assembled by mixing equimolar concentrations of four oligonucleotides with subsequent heating and annealing of the mixture. Immobile junctions are manufactured as described in ((Seeman N. C. and Kallenbach, N. R. (1994) Annu. Rev. Biophys. Biomol. Struct. 23, 53; Eichman, B. F., Vargason, G. M., Mooers B. H. M. and Ho P. S. (2000) PNAS 97, 3971) and visualized by SPM to confirm their structure.

In another embodiment the molecular quartet comprised of uninterrupted consecutive guanines, smaller DNA to ion ratio resulting in the formation of local gaps, tetraplex containing different kinds of metal ions and tetraplex containing a local distortion due to the existence of a non guanine tetrad.

By covalently linking a number of molecular wires it is possible to create nanoelectic scale switchboards or spatially resolved micro arrays. This can be achieved by introducing thiolated nucleotides at points of desired contact between 4G molecules. Oxidation of the thiol groups will result in the formation S—S covalent bonds between the two quadruplexes.

These switchboards or micro arrays are placed on a solid support and have a series of conductive organic wires operatively connected to each other. There are numerous ways known in the art as to the manner of fabricating these switchboards or micro arrays. For example, the method of DNA immobilization and patterning by electrostatic interactions with a cationic bilayer adsorbed to a self assembled monolayer (SAM) can be applied. As a result, the cationic lipids readily form layers on self assembled alkyl thiols possessing terminal carboxylic groups. DNA then can be electrostatically connected to the cationic layer (Schouten, S., Stroeve, P. and Longo, L. M. (1999) Langmuir, 15, 8133-8139).

Another method includes having DNA micro-arrays that can be produced by the combinatorial photolitographic approach developed originally by Affimetrix. Briefly, the method involves illumination through micro structured photo-mask of a chip modified with photolabile protection groups that creates selected areas to which phosphoramidate building blocks can be attached for the sake of further nucleotide attachment. (Niemeyer, C. M. and Blohm D (1999). Angew. Chem., 38 No. 19, 2865-2869).

Further methods involve utilizing glass surfaces derivatized with 3-mercaptosylane for the attachment of 5′-disulfide modified oligonucleotides via disulfide bonds (Rogers, Y. H., Baucom, P. J., Huang, Z. J., Bogdanov, V., Anderson, S. and Boyce, M. T. (1999), Analytical Biochemistry 259, 31-41). Another method involves immobilizing activated DNA on aldehyde containing polyacrylamide gels for preparation of MAGIChips, which are microarrays of gel immobilized compounds on a chip (Proudnikov, D., Timofeev, E. and Mirzabekov, A. (1998) Analytical Biochemistry 259,34-41). A number of peptides and proteins are capable of specific interaction with DNA. The mechanism of complex formation between DNA and some of the proteins and peptides include intercalation of aromatic amino acid residues into the DNA structures. (Santell, R. M. and Hsuch, J. L. (1975) Biochemistry 16, 3604-3611 and Suzuki, M. (1990) Nature 244, 562-565). Intercalation of aromatic residues results in an increase of the overall π stacking and thus increases the conductivity of the system. This enables manipulation of the conductivity of the tetra-G “wire” by changing the ratio of DNA to protein and by locally and transiently controlling DNA-protein interactions.

An additional concept includes synthesis of artificial polymers capable of forming structures similar to those of 4G-Me. These polymers comprise aromatic carbonyl containing units capable of forming stable complexes with metal ions. The above polymers include G-and T-nucleotide as well as their derivatives linked in polymers in a different fashion as compared to that in DNA. For example, one possible way of linking the nucleotides into polymers has been described. (Diederichsen, U. (1996) Angew. Chem. Int. Ed 35, 445-448 and Diederichsen, U. (1998) Bioorganic. Medical. Chem. Letters 8, 165-168) The above polymer, alanyl-RNA, consists of regular peptide strands composed of modified alanyl monomers that carry the nucleo bases at β-position as a side chain. In contrast to the negative charge carrying DNA, the alanyl-RNA molecule is neutral. By employing neutral linear polymers of G, T and their derivatives the stability of 4- or (and) poly-stranded complexes of polynucleotides can be increased with different conductive metals. The use of carbonyl-containing aromatic units different from nucleotides and the different ways of linking them in regular linear or branched chain polymers enables attainment of the requited physico-chemical and electrical properties of polymer-Me complexes needed for practical application of the complexes in electronics.

The article by Sen and Gilbert provides methods for synthesizing the major G quartet structures. The following are specific examples of such synthesis.

### EXAMPLES OF THE INVENTION

### Example 1

In general, the optimal conditions for forming G-quartet structures are at neutral pH in buffers that contain monovalent ions. In general sodium and potassium ions favor the formation of quartet structures, whereas lithium does not. Thus, where the formation of guanine-quartet structures by G-rich single-stranded DNAs is not desired, one should exclude sodium and potassium and substitute lithium, or even tetramethylammomium, in buffers.

G4-DNA: Simple Motifs (Oligomers Containing Three to Six Contiguous Guanines)

1. Dissolve the DNA oligomer (end-labeled as desired) in 10 μl of 10 mM Tris, pH 7.5, 1 mM EDTA (TE buffer) at DNA concentrations in the range of 100 μM-1 M oligomer.

2. Heat at 95° for 90 seconds in a closed Eppendorf tube and chill on ice. Spin briefly.

3. Add 10 μl of TE plus 100-500 mM NaCL or KCL, and mix by vortexing. The use of sodium or potassium is a matter of choice, but it should be noted that the potassium complexes are significantly more stable (ΔTₘ˜20°) than the sodium complexes.

4. Leave at room temperature for 30 minutes for G4-DNA to form.

The optimal electrophoresis conditions for resolving G4-DNA of simple motif oligomers from their single-stranded forms (usually the two are at equilibrium under the above buffer conditions at 20° or higher) are to chill samples on ice after incubation and to load in 6-12% nondenaturing gels run in 50 mM TE plus 5 mM KCL, at 4° (if there are five or more contiguous guanines in the oligomer, the gel can be run at room temperature). To obtain clean product bands, it is recommended that even non-denaturing gels be prerun for a period prior to loading samples. G4-DNA generally runs significantly behind the single-stranded form of the oligomer in nondenaturing gels.

### Example 2

G4-DNA: Complex Motifs (Oligomers Containing Multiple Sets of Contiguous Guanines). An important feature of the formation of complex G4-DNA motifs is that the formation is promoted by sodium and inhibited by potassium. However, the optimal conditions for the formation is a mixture of the two, usually Na:K in a 10:1 to 20:1 ratio.

1. Dissolve the DNA oligomer (end-labeled as desired) in 10 μM-1 mM oligomer.

2. Heat at 95° for 120 seconds in a closed Eppendorf tube and chill on ice. Spin briefly.

3. Add 10 μl of TE plus 1.9 M NaCL and 0.1 M KCL. Mix by vortexing and spin to collect 20 μl total solution at the bottom of the tube.

4. Draw up the solution into the central portion of a thin-walled 50 μl capillary or micropipette, using a hand-held micropipette filler.

5. Keeping one end of the micropipette attached to the filler, seal its free end by heating it to red-heat in the flame of a Bunsen burner.

6. After the sealed end has cooled, hold the micropipette by its sealed end and seal the other end.

7. Incubate the sealed capillary at 60° for 12-48 hours (for oligomers consisting of stretches of only ˜3 gaunines or containing large separations between stretches of guanines, it may be advisable to incubate at 37°, though obviously, the rate of G4-DNA formation is significantly slower).

8. After incubation, allow the capillaries to cool to room temperature. Snap off both ends of the capillaries after scoring with a glass cutter, then release the DNA solution into a clean Eppendorf tube. Spin briefly to collect liquid at the bottom of the tube.

### Example 3

In general, G4-DNAs containing upward of eight or nine guanine quartets are exceedingly stable at room temperature and are not in rapid equilibrium with their single-stranded forms. Consequently, these complexes can usually be diluted up to 10-fold without a significant change in the relative ratio of G4 to single-stranded DNA; the products can be analyzed in prerun, nondenaturing gels in 50 mM TE and at room temperature. The very slow interconversion of the G4-DNA and single-stranded forms also permits their modification in a crude mixture by such reagents as dimethyl sulfate. The partially methylated mixtures can then be separated in prerun, nondenaturing gels, eluted separately, processed with piperidine or pyrrolidone, and run on denaturing gels to study methylation protection.

The stability of G4-DNA complexes formed from sodium-only or sodium-dominant solutions can be enhanced by a subsequent addition of potassium chloride. Thus, G4-DNA formed from 1 M NaCL or 0.95 M NaCL plus 50 mM KCL can be diluted into TE plus 50 mM NaCL and 25 mM KCL, or even TE plus 10 mM KCL, for a satisfactory stability of the G4-DNA complex. It is usually also safe to ethanol precipitate G4-DNA samples from solutions containing 0.3 M sodium or potassium acetate.

### Example 4

G′2-DNA. The formation of G′2-DNA strut requires only moderate concentrations of DNA. Usually both sodium- and potassium-containing buffers are useful, except that, with sodium, one can also form a certain proportion of G4-DNA (which will not form in purely potassium buffers). Thus, one can use either sodium or potassium to form G′2-DNA when the DNA concentration is moderate (up to 1 μM; at higher concentrations of DNA, potassium alone is recommended. As with G4-DNA from simple-motif oligomers, the potassium G′2 complexes are significantly more stable than their sodium counterparts.

1. Dissolve the DNA (end-labeled as desired) in 10 μl TE buffer (see above) to 0.5-100 μM oligomer.

2. Heat in a sealed Eppendorf tube at 90° for 60 seconds. Chill on ice. Spin briefly.

3. Add 10 μl TE plus 2 M KCL. Mix by vortexing. Spin.

4. Incubate at 37° for 12-48 hours in the sealed Eppendorf tube; for relatively G-rich oligomers, it can be profitable to incubate at 60° for 12-24 hours in a sealed capillary, as described in the protocol for G4-DNA.

G′2-DNA complexes made in 1 M KCL solutions can be diluted with TE buffer to a final KCL concentration for 50 mM. Electrophoretic analysis of G′2 complexes is best achieved in prerun, nondenaturing gels in 50 mM TE plus 5-10 mM KCL at 4°; G′2 structures are generally not as stable as G4-DNA formed from the same sequence, containing fewer guanine quartets. It is useful to run the untreated single-stranded oligomer as a size control in the gel to identify the G′2 complexes, which usually run slower than the single-stranded DNA in nondenaturing gels, though not as slow as G4-DNA complexes.

### Example 5

G4′-DNA. To form the unimolecular folded-back structure G4′-DNA, one needs either a stretch of at least 28 contiguous guanines or else a minimum of four separated guanine motifs [the telomere-derived oligomers (T₄G₄)₄ and (T₂G₄)₄ are good examples of oligomers that form this structure]. Being unimolecular, the formation of G4′-DNA is independent of DNA concentration. Like all other G-quartet-containing structures, it does not form in lithium solutions but does form in sodium, potassium. Rubidium, and cesium solutions (although it should be noted that there are subtle structural differences between, e.g., the Na⁺ and K⁺ complexes, as determined by methylation probing).

To avoid generating side products of the G′2 and G4 classes, it is best to work with low concentrations (1-20 nM) of end-labeled DNA.

1. Dissolve the DNA in 5 μl TE buffer (see above) in a sealable Eppendorf tube.

2. Heat to 94° for 60 seconds. Chill on ice. Spin briefly.

3. Add 5 μl of TE plus 100 mM NaCL or KCL. Vortex to mix. Analyze G4′-DNA on nondenaturing gels run at 4° in 50 mM TE plus 50 mM NaCL or KCL, depending on which cation has been added to sample. Owing to the compactness of G4′ structures, they often move significantly ahead of the input “unfolded” oligomer during nondenaturing gel electrophoresis. As with the other quartet-containing structures, the potassium-assisted G4′ complexes are significantly more stable than their sodium counterparts.

### Example 6

G-G Base-Paired Structures. Although G-G base-paired structures do not contain G-quartets, they nevertheless are putative pathway intermediates toward the formation of G′2 and G4′ structures. The stability of G-G foldback structures (G′-DNA) of this type is not as great as that of quartet-containing structures. G′DNA structures are best defined by their greater mobility (relative to input oligomer) on nondenaturing gels. To generate G′ structures, it is important to work with low DNA concentrations and to exclude sodium and potassium salts from buffers wherever possible, substituting lithium where appropriate.

1. Dissolve the DNA (end-labeled as appropriate) at 10 nM (m.b. μM) in 10 μl TE buffer.

2. Heat at 94° for 60 seconds. Chill on ice. Spin down briefly.

Load on a nondenaturing gel run at 4° in 50 mM TE (plus 50 mM LiCl, if desired).

### Example 7

Conducting organic wires were made on the basis of G4-DNA complexes with redox active metal-ions. The molecular wire include the external surface of the molecule and a central canal, composed of linearly arranged metal ions. The metal ions are selected from a group including, but not limited, to silver, copper, zinc, iron, europium, chromium, mercury and ruthenium. Different lengths of poly-G DNA as well as poly-G that include tymidine (T) nucleotides in specific locations are made. The average distance between conducting metal ions along the molecule is controlled by the addition of redox “silent” metals (sodium, potassium) into the central core of the G-quartet together with the redox active ones. Increasing the distance between conducting metal ions results in lower electronic conduction of the G4-DNA. The combination of high and low resistance segments along the molecule enables manipulation of the conductivity of the wire and create nanodevices from these molecular wires. FIG. 7, shows a linear G4-DNA polymer structures of 0.5 microns as seen by AFM.

The experiments have shown that by employing oligonucleotides composed of 30 G (FIG. 6) a much longer (up to micron in length) wires than those obtained from 7 G-oligonucleotides. Thus, by varying the length of initial oligonucleotides and the self-assembly conditions, one can obtain G4-wires of desired length. An alternative way to produce long G4-wires is by opening long (>1000 base-pairs) double stranded Poly(G)-Poly(C) DNA at high pH with subsequent assembly of the DNA molecules at reduced pH. The assembly process is carried out in the presence of monovalent ions and leads to a rearrangement of the system in a mixture of G4-wires and single-stranded Poly(C) molecules. The latter approach will enable us to prepare continuous G4-structures of different lengths without “nicks” along the wire, that are characteristic to those made of short oligonucleotides. By varying the length of the original poly(G)poly(C) molecules we can obtain G4-structures of desired length. To produce long Poly(G)-Poly(C) molecules as well as molecules with alternating arrangement of nucleotides (A and T) in desired positions we will utilize molecular genetics approaches. The following strategies are used for the production of the above molecules: 1. Production of long double stranded DNA molecules by linking short fragments that are obtained from solid state synthesis by enzymatic reaction catalyzed by DNA ligase. 2. Using Polymerase Chain Reaction (PCR) for production of long DNA molecules. Almost any desired sequence that exists in nature can be generated using the PCR technique. This is particularly for G-rich sequences such as Poly(G)-Poly(C) that are hard to generate by chemical synthesis. Once a given sequence is generated it can be cloned using standard molecular genetics, a procedure that allows to maintain and propagate it.

The two above methods of G-wires production, i.e. self-assembly of the wires from short oligonucleotides and the formation of continuous wires from double stranded Poly(G)-Poly(C) DNA are combined to achieve organic wires of desired length. Complexes of the wires with redox active metal-ions are produced and their conduction properties are measured. The metal ions are selected from a group including but not limited to silver, copper, zinc, iron, europium, chromium, mercury and ruthenium. The central canal that is composed of linearly arranged metal ions and the electron-rich external surface of the G4-DNA molecule ensures strong electronic coupling within the structure. The latter suggests that the G4-DNA based nanowire will function as a suitable component for nanoscale electronic devices.

M-DNA wires and M-PNA wires with various redox active metal-ions are produced. The metal ions are selected from a group including: Zn²⁺, Co²⁺, or Ni²⁺. M-DNA molecules of different length, sequence and metal composition are produced. An almost linear arrangement of divalent metal ions separated by inter-base distances (˜3.4) may impose unusual conduction properties on the molecule, as was indicated by indirect optical measurements.

Direct Electrical Transport Measurements Through DNA:

Direct electrical transport measurements have been performed on: DNA molecules attached to nanofabricated metal electrodes. The measurements have been performed in the configuration shown in FIG. 8 as well as between planar arrangement of fabricated electrodes. Using electron-beam lithography, a local 30 nm narrow segment in a 100 nm wide slit is created in the SiN layer. Underetching the SiO₂ layer leads to two opposite freestanding SiN “fingers” that become the metallic nanoelectrodes after sputtering Pt through a Si mask. The middle micrograph is a scanning-electron-microscope image of the two metal electrodes and the 8 nm gap between them. Details of fabrication and trapping are described elsewhere (A. Bezryadin, C. Dekker & G. Schmid, Appl. Phys. Lett. 71, 1273 (1997) and A. Bezryadin & C. Dekker, J. Vac. Sci. Technol. B 15(4), 793 (1997)). The positioning of a DNA molecule between the electrodes was achieved by electrostatic trapping. Subsequently, a voltage of up to 5 V is applied between the electrodes. The electrostatic field polarizes a nearby molecule, which is then attracted to the gap between the electrodes due to the field gradient. The measurements have been performed on 10 nm long poly(G)-poly(C) DNA and yielded conclusive evidence that these DNA molecules are able to carry current through the molecule.

Recent measurements on a first sample, where the DNA was stabilized on the electrodes with thiol end-groups, have shown that these measurements can yield reproducible and quantitative information that enables a more detailed analysis of the experimental data.

STM/STS Measurements on DNA:

In a STM and STS study on single DNA molecules positioned on flat gold and platinum surfaces (see FIG. 9) results verify the ability to image DNA and perform tunneling spectroscopy on single DNA molecules. The current-voltage curves that are measured on single DNA molecules resemble the curves measured in the transport measurements and is therefore a good indication of our ability to extract detailed information from these measurements.

### Example 8

Electric Transport Measurements Through G4-DNA Molecules:

1) Fabrication: Planar metal (e.g. Au or Pt) electrodes are fabricated on an insulating substrate (e.g. mica or SiO₂) by standard electron-beam or optical lithography (protocols appear in any standard lithography textbook). A number of variations for the possible arrangements of the electrodes and for suitable metals and substrates are utilized (one of examples example is shown in FIG. 10). The closely separated electrodes are used for electric transport measurements through relatively short (from 10 to 50 nm long) 4G-DNA molecules.

2) Deposition: G4-DNA molecules are deposited on the electrodes such that they will connect between the electrodes and enable to measure electrical current flowing through the molecule upon application of voltage difference between the electrodes. The 4G-DNA samples prepared are dissolved in of deionized and filtered through 0.2 micron filter water. 50 μl of 1 μM solution of the DNA are applied on the surface of the chip and are left for 20 hours at mom temperature. The none-bound DNA and the salts present in the sample are removed from the surface of the chip by its extensive rinsing in a double distillate and filtrated water. The sample are dried in vacuum for at least 2 hours and subjected for the electric transport measurements.

3) Electrical transport measurements: After the deposition of the G4-DNA between the metal electrodes the sample (substrate+electrodes+G4-DNA) are bonded to a carrier chip. The chip are connected to leads that are connected to an electrometer and current/voltage measurement apparatus (e.g. Keithley). A voltage up to 10 V are applied between the electrodes and current flowing through the molecule are measured in vacuum and in He atmosphere at various conditions (voltage−10-+10 V, temperature 0.3-300 K, pressure 10⁻¹⁰-1 atm, magnetic field 0-14 T, optical excitations etc.). We expect to measure resistances in the MW range for 100 nm long 4G-DNA molecules.

What is claimed is:

1. A conductive molecular wire wherein said wire comprises a nonconductive outer organic portion surrounding an internal core of redox active ions.

2. The conductive molecular wire as in claim 1 wherein said outer organic portion comprises aligned plates of guanine tetraplexes each having a central space defining said internal core.

3. The conductive molecular wire as in claim 2 wherein said outer organic portion includes aligned plates of guanine tetraplexes each having a central space defining said internal core, wherein at least some of said internal cores chelating said redox active ions.

4. The conductive molecular wire as in claim 1, wherein the outer organic portion comprises a DNA, RNA, M-DNA, or PNA backbone.

5. The conductive molecular wire as in claim 3 wherein said redox active ions are selected from the group consisting essentially of silver, copper, iron, europium, chromium, mercury, and ruthenium.

6. The conductive molecular wire as in claim 3 further comprising one or more spacing means disposed between at least one of said plates of tetraplexes for increasing resistance to electron flow through said wire.

7. The conductive molecular wire as in claim 6 wherein said spacing means includes one or more plates of non-guanine nucleotides disposed between at least two of said plates of guanine tetraplexes.

8. The conductive molecular wire as in claim 1 comprising one or more operative interconnections to other conductive molecular wires.

9. The conductive molecular wire as in claim 8 wherein said interconnections include covalent linking means for covalently connecting an end plate of at least one of said tetraplexes to a tetraplex of a second conductive molecular wire.

10. The conductive molecular wire as in claim 8 wherein said lining means includes oxidized thiol groups.

11. The conductive molecular wire as in claim 1 including one or more switching means for controlling electron flow between said redox active ions.

12. The conductive molecular wire as in claim 11 wherein said switching means includes molecules capable of intercalating between adjacent plates of guanine tetraplexes.

13. A method for producing a conductive molecular wire by spontaneously forming guanine tetraplexes hydrogen bonded together and stabilizing the wire through coordinated redox active ions disposed in a core of at least some of the tetraplexes.

14. The method according to claim 13, including the further step of selectively increasing the conductivity of the wire.

15. The method according to claim 14, wherein said increasing step is further defined as increasing the ratio of redox active ions to tetraplexes in the wire.

16. The method according to claim 14, wherein said increasing step is further defined as incorporating an increased ratio of redox active ions having higher conductivity into the wire.

17. The method according to claim 13, including the step of increasing resistance to conductivity of the wire.

18. The method according to claim 17, wherein said step of increasing the resistance is further defined as selectively incorporating nonconductive metal into at least some of said tetraplexes.

19. The method according to claim 18, wherein said step of increasing the resistance is further defined as selectively incorporating nonconductive metal into at least some of said tetraplexes selected from the group including sodium and potassium.

20. The method according to claim 13, further including the step of forming peptide strands of guanine.

21. A transistor comprising a conductive molecular wire wherein said wire includes a nonconductive outer organic portion surrounding an internal core of redox active ions.

22. A switch comprising electron transport means incorporated into the conductive organic wire for controlling electron flow.

23. The switch as in claim 20 being selected from the group including electro-optical switches and phototransitors.

24. The switch as in claim 20 wherein said electro-optical switch and phototransistor includes guanine tetrad complexes coordinating metals capable of changing redox potential upon iridation of light.

25. The switch as in claim 21 wherein said coordinated metals are selected essentially from the group including zinc and ruthenium.

26. A method of switching on and off current through an molecular wire by activating an electron transport control mechanism through light irridation of said electron transport control mechanism.

27. The method as in claim 26 further defined as changing the redox potential of an ion coordinated in a guanine tetrad complex.

28. The method as in claim 27 wherein said changing step is further defined as mediating the ion to change its redox potential.

29. A mirco-array comprising a support and series of conductive organic wires operatively connected to each other disposed on said support.
